(19) United States
(12) Patent Application Publication
(10) Pub. No.: US 2006/0280698 A1
(43) Pub. Date: Dec. 14, 2006

(54) System for treating conditions of the periodontium

(76) Inventors: Subhash Gupta, Leominster, MA (US); Dennis Flanagan, Mansfield Center, CT (US)

Correspondence Address: KRIEGSMAN & KRIEGSMAN, 665 Franklin Street, Framingham, MA, 01702, US

(21) Appl. No.: 11/152,499

(22) Filed: Jun. 14, 2005

(51) Int. Cl. A61K 8/96; A61K 36/18

(52) U.S. Cl. 424/50; 424/725

(57) ABSTRACT

A system for treating conditions of the periodontium, such as gingivitis and periodontitis, includes an Ayurvedic medicinal solution and an applicator for delivering the solution to the periodontium. The Ayurvedic medicinal solution utilizes herbal extracts to break-down bacteria which can inflame gum tissue. In one embodiment, the solution comprises approximately 1 gram of triphala extract for every 10 ml of glycerine. In another embodiment, the solution comprises approximately 1 gram of amla extract for every 10 ml of glycerine. The applicator for delivering the solution to the periodontium may either be in the form of a cotton swab-type wand, a pipette or a spray dispenser.

### BACKGROUND OF THE INVENTION

The present invention relates generally to the dental industry and more particularly to the treatment of conditions of the periodontium such as gingivitis and periodontitis.

The periodontium (also referred to herein as gum tissue) is the tissue within a person's mouth that surrounds and supports teeth. Specifically, referring generally to FIG. 1, there is shown a fragmentary, side plan view of a pair of teeth 11-1 and 11-2 which are surrounded and supported by gum tissue 13.

As can be appreciated, it has been found that the build-up of plaque in a patient's mouth can create a number of medical conditions of the periodontium. For example, the build-up of plaque can result in the condition of gingivitis. Gingivitis is a relatively common condition which is characterized by inflammation and/or bleeding of the gum tissue as well as by bad breath. It should be noted that, if improperly treated, gingivitis may lead to more serious conditions, such as periodontal disease.

Periodontal disease is a form of advanced gum disease which is characterized by considerable inflammation of gum tissue. If improperly treated, periodontal disease can cause teeth to loosen and, over time, ultimately fall out, which is highly undesirable.

Accordingly, numerous treatments are presently available for treating various medical conditions of the periodontium which result from the build-up of plaque in a patient's mouth.

As an example, in U.S. Pat. No. 6,106,286 to Subhash Gupta, which is incorporated herein by reference, there is disclosed a dental loop for delivering Coenzyme Q10 to the periodontium by direct physical contact therewith, and a method using such a loop. The loop is a loop of plain gut defining a series of pockets for carrying a medicament. Coenzyme Q10 is placed within the pockets, so that it is available directly at the site to which said loop is applied. The loop is slipped over a tooth and placed against the periodontium, and left in place for sufficient time for the Q10 to dissolve and act, and for the loop itself to dissolve.

Traditional medications used in the treatment of conditions of the periodontium are typically created in a laboratory environment using complex chemical solutions and, as a result, are often relatively expensive to purchase, which is highly undesirable.

Due to the escalating costs of such medications, herbal medications (i.e., plant-derived extracts which exhibit medicinal properties) are presently being considered with respect to the treatment of a wide variety of different diseases. As can be appreciated, herbal treatments have been found to be a desirable alternative to conventional medications because herbal medications are both relatively inexpensive to manufacture and subject its users to minimal, if any, known side-effects.

Ayurvedic medicine is one form of herbal medicine which is well-known in the art. In India, ayurvedic medicine is commonly used in periodontal therapy to control bleeding and reduce inflammation of the periodontium. Herbs that have been found to be useful in treating gingivitis (and other known conditions of the periodontium) include chamomile, echinacea, green tea, peppermint, sage, clove and myrrh.

### SUMMARY OF THE INVENTION

It is an object of the present invention to provide a new and improved system for the treatment of conditions of the periodontium, such as gingivitis and periodontitis.

It is another object of the present invention to a system of the type as described above which is inexpensive to manufacture.

It is yet another object of the present invention to provide a system as described above which subjects its user to minimal side effects.

It is yet still another object of the present invention to provide a system as described above which utilizes herbal medicines.

It is even still another object of the present invention to provide a system as described above which provides an easy-to-use means for delivering the medicine to the periodontium.

Therefore, according to one feature of the present invention, there is provided a system for treating conditions of the periodontium, such as gingivitis and periodontitis, said system comprising, (a) a medicinal solution comprising triphala extract and glycerine, and (b) an applicator for delivering the medicinal solution to the periodontium.

According to another feature of the present invention, there is provided a solution for treating conditions of the periodontium, said solution comprising (a) triphala extract, and (b) glycerine.

According to another feature of the present invention, there is provided a system for treating conditions of the periodontium, such as gingivitis and periodontitis, said system comprising (a) a medicinal solution comprising amla extract and glycerine, and (b) an applicator for delivering the medicinal solution to the periodontium.

According to another feature of the present invention, there is provided a solution for treating conditions of the periodontium, said solution comprising (a) amla extract, and (b) glycerine.

Various other features and advantages will appear from the description to follow. In the description, reference is made to the accompanying drawings which form a part thereof, and in which is shown by way of illustration, various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is therefore, not to be taken in a limiting sense, and the scope of the present invention is best defined by the appended claims.

### BRIEF DESCRIPTION OF THE DRAWINGS

In the drawings wherein like reference numerals represent like parts:

FIG. 1 is a fragmentary, side plan view of a pair of teeth which are surrounded and supported by gum tissue;

FIG. 2 is a partially exploded, front perspective view, broken away in part, of a first embodiment of a system for treating conditions of the periodontium, the system being constructed according to the teachings of the present invention;

FIG. 3 is a front perspective view, broken away in part, of a second embodiment of a system for treating conditions of the periodontium, the system being constructed according to the teachings of the present invention; and

FIG. 4 is a partially exploded, front perspective view, broken away in part, of a third embodiment of a system for treating conditions of the periodontium, the system being constructed according to the teachings of the present invention.

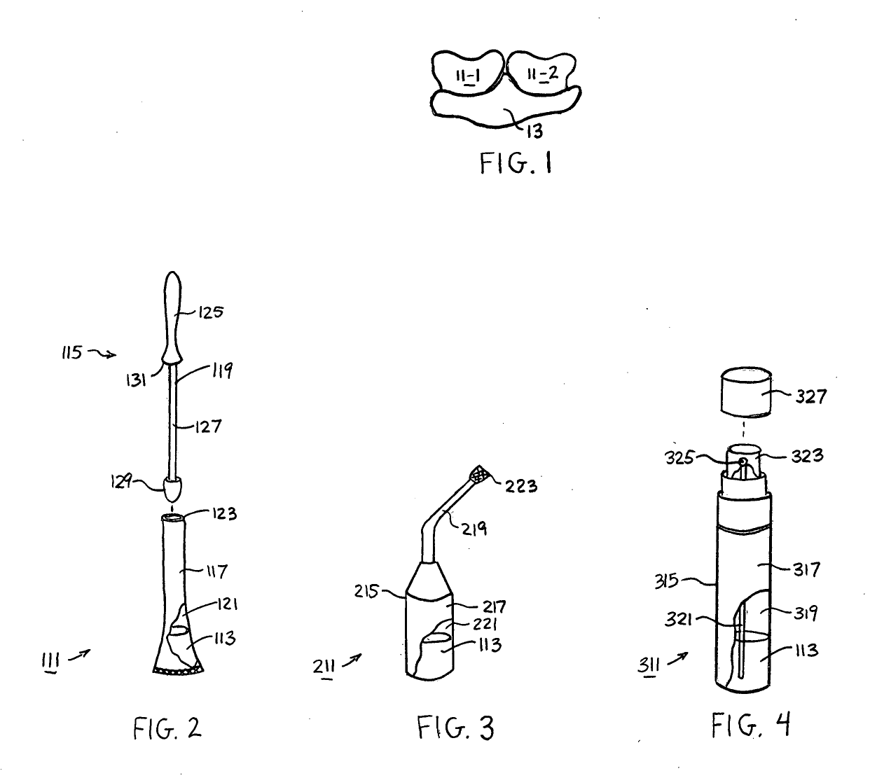

### DETAILED DESCRIPTION OF THE PREFERRED EMBODIMENT

Referring now to FIG. 2, there is shown a first embodiment of a novel system for treating conditions of the periodontium, such as gingivitis and periodontitis, the system being constructed according to the teachings of the present invention and identified generally by reference numeral 111.

System 111 includes a medicinal solution 113 and an applicator 115 for delivering medicinal solution 113 to the periodontium.

Medicinal solution 113 is an Ayurvedic medicinal solution which is designed to break-down the common forms of bacteria which can inflame gum tissue. As will be described in detail below, medicinal solution 113 may be represented in either of the two following forms:

(1) Triphala Solution: The triphala solution preferably comprises approximately 1 gram of triphala extract for every 10 ml of glycerine. However, it is to be understood that the concentration of triphala relative to glycerine may be adjusted without departing from the spirit of the present invention.

It should be noted that triphala is an Ayurvedic name for the herbal mixture derived from the acrid fruits of the following three plants, the herbals being mixed in equal parts: (1) Terminalia Chebula (Ayurvedic name: Haritaki); (2) Terminalia Belerica (Ayurvedic name: Bibhitaki); and (3) Embilica Officinalis (Ayervedic name: Amalaki or Amla). Together, the botanical blend of the three herbal extracts identified above creates a powder which is yellowish-green to greenish-brown in color.

(2) Amla Solution: The amla solution preferably comprises approximately 1 gram of amla extract for every 10 ml of glycerine. However, it is to be understood that the concentration of amla relative to glycerine may be adjusted without departing from the spirit of the present invention.

As noted above, amla (also commonly referred to as amalki) is the Ayurvedic name for the herbal derived from the acrid fruit of the Embilic Officinalis plant. Amla extract is typically in the form of a brownish powder.

As seen most clearly in FIG. 2, applicator 115 comprises a tube 117 configured to retain a supply of medicinal solution 113 and an applicator wand 119 for delivering a supply of solution 113 to the periodontium, as will be described further below.

Preferably, tube 117 is a generally cylindrical plastic member which is crimped at one end (e.g., by means of ultrasonic welding) so as to define a partially enclosed cavity 121 which is accessible through an open top end 123. It should be noted that cavity 121 is preferably sized and shaped to retain an adequate supply of solution 113 (e.g., 1 ml, 2 ml, etc.).

Applicator wand 119 includes an elongated plastic handle 125 and a stem 127 which are connected together. The free end of stem 127 is provided with an enlarged cotton tip, or swab, 129 which is constructed to retain a small amount (i.e., a dab) of solution 113 thereon, as will be described further below.

Applicator wand 119 is sized and shaped such that stem 127 and tip 129 may be inserted through open end 123 of tube 117 and down into cavity 121. Positioned as such, swab 129 becomes submerged in solution 113. Preferably, handle 125 is provided with an outwardly extending flange 131 which is initially secured to tube 117 at open end 123 (e.g., through ultrasonic welding) to ensure that solution 113 is retained within applicator 115 during shipping.

In use, applicator 115 may be used in the following manner to deliver a supply of solution 113 to the periodontium. Specifically, as noted above, flange 131 of applicator wand 119 is initially secured to tube 117 at open end 123 to retain solution 113 within applicator 115 during shipping. In order to access solution 113, the seal between flange 131 and tube 117 is broken (e.g., by wiggling or bending applicator wand 119). With the seal broken, applicator wand 119 is withdrawn from tube 117.

With applicator wand 119 withdrawn from tube 117, it should be noted that a small supply of solution 113 is absorbed onto cotton tip 129. As a result, in order to spot apply (i.e., dab) a small quantity of solution 113 onto the periodontium, the user grasps handle 125 and orientates applicator wand 119 such that the cotton tip 129 is drawn into direct contact with the patient's gum line. It should be noted that, in order to treat an extended area of the periodontium, the user may be required to repeatedly submerge cotton tip 129 back into the solution 113 contained within tube 117. Preferably, the above-described treatment is performed once a day for three weeks.

It should be noted that system 111 is not limited to the use of applicator 115 to deliver medicinal solution 113 to the periodontium. Rather, it is to be understood that alternate types of applicators may be used to deliver medicinal solution 113 to the periodontium without departing from the spirit of the present invention.

As an example, referring now to FIG. 3, there is shown a second embodiment of a system for treating conditions of the periodontium, the system being constructed according to the teachings of the present invention and identified generally by reference numeral 211.

System 211 includes medicinal solution 113 and an applicator 215 for delivering medicinal solution 113 to the periodontium. System 211 differs from system 111 only in the fact that system 211 utilizes applicator 215, rather than applicator 115, to deliver solution 113 to the periodontium.

Applicator 215 is in the form of a unitary plastic pipette which comprises a compressible bulb 217 that is connected to a bent length of tubing 219.

Bulb 217 is shaped to define a partially enclosed cavity 221 which is accessible only by means of tubing 219. It should be noted that cavity 221 is preferably sized and shaped to retain an adequate supply of solution 113 (e.g., 1 ml, 2 ml, etc.).

Tubing 219 extends out at an angle from an opening formed in the top surface of bulb 217. The free end of tubing 219 is preferably enclosed with a stop 223 which may be formed, for example, by crimping the free end of tubing 219. Stop 223 is preferably designed to be severed from tubing 219 so as to create an opening in applicator 215 through which solution 113 may exit, as will be described further below.

In use, applicator 215 may be used in the following manner to deliver a supply of solution 113 to the periodontium. Specifically, when ready for use, stop 223 is severed from the remainder of tubing 219 (e.g., using a suitable cutting instrument, such as a knife or scissors), thereby creating an opening in the tip of tubing 219 through which solution 113 may exit.

Handling applicator 215 by bulb 217, the user manipulates applicator 215 such that the opening in the tip of tubing 219 is disposed in contact with (or in close proximity to) the area of the periodontium in need of medicinal solution 113. The user then compresses bulb 217 which, in turn, expresses a stream of solution 113 out from tubing 219 and onto the area of the periodontium in need of treatment. In order to treat additional areas of the periodontium, applicator 215 is repositioned and bulb 217 is compressed as needed to deliver solution 113 to the gum line.

As another example, referring now to FIG. 4, there is shown a third embodiment of a system for treating conditions of the periodontium, the system being constructed according to the teachings of the present invention and identified generally by reference numeral 311.

System 311 includes medicinal solution 113 and an applicator 315 for delivering medicinal solution 113 to the periodontium. System 311 differs from system 111 only in the fact that system 311 utilizes applicator 315, rather than applicator 115, to deliver solution 113 to the periodontium.

Applicator 315 is in the form of a spray dispenser and includes a rigid and durable container 317 that is shaped to define a partially enclosed cavity 319. It should be noted that cavity 319 is preferably sized and shaped to retain an adequate supply of solution 113 (e.g., 7.5 ml).

One end of an elongated delivery tube 321 is disposed into solution 113. The other end of delivery tube 321 is connected to a pump 323 which is coupled to container 317. As will be described further below, depression of pump 323 draws solution 113 into delivery tube 321 and ultimately causes solution 113 to exit through a valve 325 provided in pump 323, solution 113 exiting valve 325 as a fine mist. A removable cap 327 may be provided to cover pump 323 when applicator 313 is not in use.

In use, applicator 315 may be used in the following manner to deliver a supply of solution 113 to the periodontium. Specifically, when ready for use, cap 327 is removed from container 317 so as to expose pump 323. Handling applicator 315 by container 317, the user manipulates applicator 315 such that the valve 325 is directed towards the area of the periodontium in need of medicinal solution 113. With the applicator 315 positioned as such, the user then depresses pump 323. The depression of pump 323 extracts a supply of solution 113 up into delivery tube 321 and ultimately out through valve 325 as a fine mist which coats the area of the periodontium in need of treatment. In order to treat additional areas of the periodontium, applicator 315 is repositioned and pump 323 is depressed as needed to deliver solution 113 to the gum line.

The embodiments shown in the present invention are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A system for treating conditions of the periodontium, such as gingivitis and periodontitis, said system comprising,
(a) a medicinal solution comprising,
(i) triphala extract, and
(ii) glycerine, and
(b) an applicator for delivering the medicinal solution to the periodontium.

2. The system as claimed in claim 1 wherein the medicinal solution includes approximately 1 gram of triphala for every 10 ml of glycerine.

3. The system as claimed in claim 2 wherein the applicator comprises a cotton swab that is formed onto one end of an applicator wand.

4. The system as claimed in claim 2 wherein the applicator is in the form of a pipette.

5. The system as claimed in claim 2 wherein the applicator is in the form of a spray dispenser.

6. A solution for treating conditions of the periodontium, said solution comprising:
(a) triphala extract, and
(b) glycerine.

7. The solution as claimed in claim 6 wherein the solution includes approximately 1 gram of triphala for every 10 ml of glycerine.

8. A system for treating conditions of the periodontium, such as gingivitis and periodontitis, said system comprising,
(a) a medicinal solution comprising,
(i) amla extract, and
(ii) glycerine, and
(b) an applicator for delivering the medicinal solution to the periodontium.

9. The system as claimed in claim 8 wherein the medicinal solution includes approximately 1 gram of amla for every 10 ml of glycerine.

10. The system as claimed in claim 9 wherein the applicator comprises a cotton swab that is formed onto one end of an applicator wand.

11. The system as claimed in claim 9 wherein the applicator is in the form of a pipette.

12. The system as claimed in claim 9 wherein the applicator is in the form of a spray dispenser.

13. A solution for treating conditions of the periodontium, said solution comprising:
(a) amla extract, and
(b) glycerine.

14. The solution as claimed in claim 13 wherein the solution includes approximately 1 gram of amla for every 10 ml of glycerine.
